(19) United States
(12) Patent Application Publication
(10) Pub. No.: US 2021/0000492 A1
(43) Pub. Date: Jan. 7, 2021

(54) SELF-SUPPORTING SURGICAL GUIDE

(71) Applicant: MOR RESEARCH APPLICATIONS LTD., Tel Aviv (IL)

(72) Inventors: Nimrod ROZEN, Tel Aviv (IL); Aharon AMIR, Tel Aviv (IL)

(21) Appl. No.: 16/975,953

(22) PCT Filed: Feb. 27, 2019
(86) PCT No.: PCT/IB2019/051562

§ 371 Date: Aug. 26, 2020

Related U.S. Application Data
(60) Provisional application No. 62/636,380, filed on Feb. 28, 2018.

(51) Int. Cl. A61B 17/17; A61B 17/16

(52) U.S. Cl. CPC A61B 17/1785; A61B 17/1604

(57) ABSTRACT

A device for guiding a surgical instrument at a surgical site, comprising: at least one guide channel sized and fitted to slidingly accommodate a surgical instrument, said guide channel defining a guide longitudinal axis; and at least one guide support leg coupled to said guide and defining a support leg longitudinal axis, wherein an angle (a) of said guide longitudinal axis in respect to a surface of a surgical site is defined by at least one of (i) a length (L) of said support leg, and (ii) an angle (β) between said guide longitudinal axis and said support leg longitudinal axis.

### FIELD OF THE INVENTION

The invention relates to the field of surgical tool guides, and more specifically to the field of orthopedic surgical tool guides.

### BACKGROUND

Osteotomy is a surgical procedure for resecting, cutting, removing, and reshaping bones with the help of a special chisel, or osteotome. An osteotome may be used free-hand, such that the surgeon guides the osteotome with one hand and uses the other hand to strike and propel the osteotome, using a mallet or a sliding weight. This approach carries several disadvantages in applications such as rhinoplasty, in which precise control of the osteotome is of paramount importance. For example, when the chisel is struck by the mallet or sliding weight, it can deviate or become misaligned, which may result in a non-optimal treatment of the particular bone.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

According to a first aspect of the present invention, there is provided a device for guiding a surgical instrument at a surgical site, comprising: at least one guide channel sized and fitted to slidingly accommodate a surgical instrument, the guide channel defining a guide longitudinal axis, and at least one support leg coupled to the guide channel and defining a support leg longitudinal axis, wherein an angle (α) of the guide longitudinal axis in respect to a surface of a surgical site is determined by at least one of (i) a length (L) of the at least one support leg, and (ii) an angle (β) between the guide longitudinal axis and the at least one support leg longitudinal axis.

In some embodiments, the at least one support leg is coupled to the guide channel through a coupling configured to allow angular adjustment of the at least one support leg in relation to the guide channel.

In some embodiments, the coupling has a locked state in which said at least one support leg is locked in a desired position relative to the guide channel, and an unlocked state in which the at least one support leg is able to be moved relative to the guide channel about the coupling.

In some embodiments, the coupling is selected from the group consisting of: a pivotable joint, a ball joint, and a universal joint.

In some embodiments, the coupling comprises a shaft disposed transversely in relation to the guide channel, wherein the shaft is rotatable about its longitudinal axis, and is lockable in a fixed position to prevent rotation of said shaft relative to said guide channel.

In some embodiments, the length (L) of the at least one support leg is adjustable. In some embodiments, at least one support leg is a telescopic support leg.

In same embodiments, the coupling is configured to slidingly receive the at least one support leg and to slide along at least a portion of the length (L) of the at least one support leg, wherein the coupling is lockable is a position along the at least a portion of the length (L) of the at least one support leg.

In some embodiments, the device further comprises a handle assembly. In some embodiments, the handle assembly is detachably coupled to the guide channel. In some embodiments, the handle assembly comprises an elongated grip portion oriented along the guide longitudinal axis. In some embodiments, the device contacts the bone at at least three points of contact. In some embodiments, at least two of the three points of contact are on different sides of the surgical site.

In some embodiments, the device is made from one or more materials selected from the group of materials consisting of: stainless steel alloy and polymer. In some embodiments, the device is made of polymer, and at least a portion of the guide channel is coated in a protective layer configured to resist abrasion.

In some embodiments, the device further comprises at least one support arm rigidly coupled to the guide channel. In some embodiments, the at least one support arm extends distally from a side of a distal end of the guide channel along the guide longitudinal axis. In some embodiments, the device further comprises a transverse projection extending laterally from the at least one support arm.

In some embodiments, the device further comprises two opposed elongated support arms extending distally from opposite sides of a distal end of the guide channel along the guide longitudinal axis. In some embodiments, the device further comprises a bridge disposed transversely between the two support arms, wherein the bridge is attached to a top portion of each of the support arms.

In some embodiments, the guide channel comprises a pair of opposing grooves extending along the guide longitudinal axis, wherein the grooves are configured to slideably engage corresponding side edges of the surgical instrument. In some embodiments, a transverse distance between the pair of grooves is adjustable. In some embodiments, at least one of a height and a depth of each of the grooves is adjustable.

In some embodiments, the guide channel is at least in part a hollow rectangular sheath. In some embodiments, the guide channel is detachably coupled to the device.

In some embodiments, the guide channel is sized and fitted to slidingly accommodate a first surgical instrument and a second surgical instrument, wherein each of the first surgical instrument and the second surgical instrument are disposed along the guide longitudinal axis.

In some embodiments, the guide channel comprises a pair of opposing two-stepped grooves extending along the guide longitudinal axis, wherein a first step of the pair of opposing two-stepped grooves is sized and fitted to slidingly accommodate the first surgical instrument, and a second step of the pair of opposing two-stepped grooves is sized and fitted to slidingly accommodate the second surgical instrument.

In some embodiments, the first surgical instrument is selected from the group consisting of: osteotome, chisel, and gouge.

In some embodiments, the second surgical instrument is a septal stabilizer. In some embodiments, the septal stabilizer is configured for stabilizing a nasal septum during open rhinoplasty, wherein the septal stabilizer comprises an elongated planar strip having a proximal end and a distal end, wherein the distal end has a centrally-located slot configured to receive at least a portion of the nasal septum. In some embodiments, the opening of the slot broadens distally. In some embodiments, the septal stabilizer further comprises a stop portion configured to prevent at least a portion of the septal stabilizer from sliding into a proximal opening of the guide channel.

In another aspect of the present invention, there is provided a device for guiding a surgical instrument at a surgical site, comprising: a handle, a guide channel sized and fitted to slidingly accommodate a surgical instrument, the guide channel defining a guide longitudinal axis; two opposed elongated support arms extending distally from opposite sides of a distal end of the guide channel along the guide longitudinal axis, a bridge disposed transversely between the support arms, wherein the bridge is attached to a top portion of each of the support arms, and a pair of support legs disposed on either side of the body, wherein each of the support legs is pivotable about an axis that is transverse to the guide longitudinal axis, and a length of each support leg is adjustable.

In a another aspect of the present invention, there is provided a method for performing rhinoplasty comprising: providing a guide channel sized and fitted to slidingly accommodate a surgical instrument, wherein the guide channel has at least two support arms and at least one support leg, positioning said guide channel at a rhinoplasty surgical site such that each of the support arms engages a point on a frontal bone of a subject, wherein the points are on either side of a nasal skeleton of the subject, establishing a desired angle (α) of a guide longitudinal axis of the guide channel in respect to a surface of the surgical site, by adjusting at least one of (i) a length (L) of the at least one support leg, and (ii) an angle (β) between the guide longitudinal axis and a longitudinal axis of the at least one support leg, urging the at least one support leg against a surface of the bone, sliding a surgical instrument into the guide channel until a cutting edge of the surgical instrument is exposed at a distal end of the guide channel, and advancing the surgical instrument through the guide channel to resect at least a portion of the nasal skeleton.

In some embodiments, the surgical instrument is selected from the group consisting of: osteotome, chisel, and gouge.

In some embodiments, the step of resecting comprises resecting, in a single pass of the surgical instrument, a cartilaginous portion of the nasal skeleton and a bony portion of the nasal skeleton.

In some embodiments, the rhinoplasty is open rhinoplasty or closed rhinoplasty.

In some embodiments, the at least one support leg is coupled to the guide channel through a coupling configured to allow angular adjustment of the at least one support leg in relation to the guide channel. In some embodiments, the coupling has a locked state in which the at least one support leg is locked in a desired position relative to the guide channel, and an unlocked state in which the at least one support leg is able to be moved relative to the guide channel about the coupling. In some embodiments, the step of establishing further comprises the steps of: unlocking the coupling; adjusting said angle (β) between the guide longitudinal axis and the longitudinal axis of the support leg; and locking the coupling.

In some embodiments, the coupling is selected from the group consisting of: a pivotable joint, a ball joint, and a universal joint.

In some embodiments, the coupling comprises a shaft disposed transversely in relation to the guide channel, wherein the shaft is rotatable about its longitudinal axis, and is lockable in a fixed position to prevent rotation of the shaft relative to the guide channel. In some embodiments, the step of establishing further comprises the steps of: unlocking said shaft, adjusting the angle (β) between the guide longitudinal axis and the longitudinal axis of the support leg, by rotating the shaft to a desired position relative to the guide channel, and locking the shaft.

In some embodiments, the length (L) of the at least one support leg is adjustable.

In same embodiments, the coupling is configured to slidingly receive the at least one support leg and to slide along at least a portion of said length (L) of the at least one support leg, wherein the coupling is lockable is a position along the at least a portion of the length (L) of the at least one support leg. In some embodiments, the step of establishing further comprises the steps of: unlocking the coupling, adjusting the length (L) of the at least one support leg by sliding the coupling to a desired position along the length of the support leg, and locking the coupling.

In some embodiments, the guide channel has two support legs disposed on opposite sides of the guide channel, and the step of establishing further comprises establishing a desired angle (δ) of a guide transverse axis of the guide channel in respect to a surface of the surgical site, by adjusting separately the length (L) of each of the two support legs.

In some embodiments, the two support arms are two opposed elongated support arms extending distally from opposite sides of a distal end of said guide channel along the guide longitudinal axis. In some embodiments, the two support arms further comprise a bridge disposed transversely between said two support arms, wherein the bridge is attached to a top portion of each of the support arms. In some embodiments, the step of positioning further comprises positioning said support bridge against one of: (i) a top portion of the nasal skeleton, and (ii) a glabella of the subject.

In some embodiments, the guide channel comprises a pair of opposing grooves extending along opposing sides of the guide longitudinal axis, wherein the grooves are configured to slidably engage corresponding side edges of the surgical instrument. In some embodiments, a transverse distance between the pair of grooves is adjustable. In some embodiments, at least one of a height and a depth of each of the grooves is adjustable.

In some embodiments, the guide channel is sized and fitted to slidingly accommodate a first surgical instrument and a second surgical instrument, wherein each of said first surgical instrument and the second surgical instrument is disposed along the guide longitudinal axis.

In some embodiments, the second surgical instrument is a septal stabilizer configured for stabilizing a nasal septum of the subject during rhinoplasty, the septal stabilizer comprising an elongated planar strip having a proximal end and a distal end, said distal end having a centrally-located slot configured to receive at least of portion of the nasal septum. In some embodiments, the opening of the slot broadens distally. In some embodiments, the septal stabilizer further comprises a stop portion configured to prevent at least a portion of the septal stabilizer from sliding into a proximal opening of the guide channel. In some embodiments, the step of sliding further comprises sliding the septal stabilizer into the guide channel underneath the first surgical instrument, such that at least a portion of the nasal septum is received within the slot.

In some embodiments, the guide channel comprises a pair of opposing two-stepped grooves extending along opposite sides of the guide longitudinal axis, wherein a first step of the pair of opposing two-stepped grooves is sized and fitted to slidingly accommodate the first surgical instrument, and a second step of the pair of opposing two-stepped grooves is sized and fitted to slidingly accommodate the second surgical instrument.

In some embodiments, there is further provided a handle assembly, wherein the handle assembly is detachably coupled to the guide channel. In some embodiments, the handle assembly comprises an elongated grip portion oriented along the guide longitudinal axis.

According to yet another aspect of the present invention there is provided a method for performing rhinoplasty including positioning a guide channel having at one support arm and at least one support leg at a rhinoplasty surgical site, urging the support arm against a surface of the bone, to one side of the surgical site, urging the at least one support leg against a surface of the bone to a second side of the surgical site and fixing the guide channel at an angle (α) between a longitudinal axis of the guide channel and the surface of the bone.

In some embodiments, the guide channel includes at least two arms positioned on either side of the surgical site. In some embodiments, the method includes sliding a surgical tool along the channel and reducing both cartilage and bone tissues with the surgical tool. In some embodiments, the support arms and support legs contact bone at at least three points of contact.

According to an aspect of some embodiments of the present invention, there is provided a system including a device for guiding a surgical instrument at a surgical site including: at least one guide channel sized and fitted to slidingly accommodate a surgical instrument, the guide channel defining a guide longitudinal axis, and at least one support leg coupled to the guide channel and defining a support leg longitudinal axis, wherein an angle (α) of the guide longitudinal axis in respect to a surface of a surgical site is determined by at least one of (i) a length (L) of the at least one support leg, and (ii) an angle (β) between the guide longitudinal axis and the at least one support leg longitudinal axis, a control unit (implemented, for example, by circuitry), and at least one sensor in communication with the control unit, wherein the at least one sensor is positioned to determine at least one of a location, a position, and an orientation of the guide channel of the device in relation to at least one reference point.

In some embodiments, at least one sensor is positioned on the device. In some embodiments, at least one sensor is positioned at the surgical site.

In some embodiments, at least one reference point includes a marker associated with the at least one sensor, and wherein the determining includes measuring one of a distance, direction, alignment, orientation, and angle between the at least one sensor and the marker.

In some embodiments, at least one marker is configured to be positioned on the device. In some embodiments, at least one marker is configured to be positioned at the surgical site.

In some embodiments, the control unit is configured to model a surgical procedure with respect to the surgical site using the device based, at least in part, on an image of the surgical site received by the control unit, and wherein the modelling determines a modeled location, position, and orientation of the device in relation to the surgical site.

In some embodiments, the control unit is further configured to compare between the determined location, position, and orientation of the device and the modeled position, location, and orientation.

In some embodiments, the control unit is further configured to alert a user of a deviation between the determined and the modeled location, position, and orientation of the device, if the deviation exceeds a predetermined threshold.

In some embodiments, the image is one or more of a digital image, x-ray image, Magnetic resonance imaging (MRI) scan, and computed tomography (CT) scan. In some embodiments, the control unit further includes at least one of a display, a processing module and a user interface module. In some embodiments, the alert is one or more of an auditory alert, a visual alert, a textual alert, and a haptic alert.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-1C illustrate 3D representations of an osteotome guide device, according to certain embodiments of the present disclosure;

FIGS. 2A-2D illustrate several views of an osteotome guide body, according to an embodiment of the present disclosure;

FIGS. 3A-3C illustrate several views of an osteotome guide handle assembly, according to an embodiment of the present disclosure;

FIG. 4A illustrates a side view of an adjustable osteotome guide, according to certain embodiments of the present disclosure;

FIGS. 4B-4E illustrate various adjustment possibilities provided by an adjustable osteotome guide, according to certain embodiments of the present disclosure;

FIG. 4F illustrates back views of an adjustable osteotome guide, according to certain embodiments of the present disclosure;

FIGS. 5A-5C illustrate an osteotome guide device with an osteotome, according to certain embodiments of the present disclosure;

FIGS. 6A-6D illustrate a septal stabilizer for use with an osteotome guide, according to certain embodiments of the present disclosure;

FIGS. 7A-7D illustrate a septal stabilizer for use with an osteotome guide according to certain embodiments of the present disclosure;

FIG. 8 illustrates an osteotome guide device in situ, according to an embodiment of the present disclosure;

FIG. 9 is a block diagram of a method for using an osteotome guide, according to an embodiment of the present disclosure; and

FIG. 10 is an osteotome guide system according to some embodiments of the present disclosure.

### DETAILED DESCRIPTION

Disclosed herein are a device and a method for accurately orienting and guiding a medical instrument or tool during medical procedures. The present device provides a self-supporting guide for controllable application of the instrument along a predetermined path, without the need for manual control of the guide, as the instrument is being propelled under impact.

The following discussion will be directed at an exemplary application of the present invention in the area of nasal bridge reduction, where the medical instrument is an osteotome or chisel. However, other applications of the present invention may be considered, in medical procedures such as tibial osteotomy.

Reduction of the nasal bridge may be performed during open or closed rhinoplasty. Open rhinoplasty involves making a trans-columella incision and peeling back the skin to expose the nasal skeleton structure. In closed rhinoplasty, all the procedural incisions are performed within the nose, without cutting the columella. The surgeon then marks the planned lowering of the nasal hump, which comprises the cartilaginous hump and the nasal bone hump. Typically, the cartilaginous hump and nasal bone hump are removed in separate steps, because the sudden change in tissue density and hardness at the junction between the two parts can cause destabilization or deflection (e.g., a “jump”) of the cutting tool, which may lead to undesirable results. Accordingly, in many cases, the surgeon first excises the cartilaginous section of the hump using, e.g., a scalpel and/or scissors. The surgeon then separately reduces the bony section of the hump using an osteotome, a rasp, or a combination thereof. This two-step procedure requires the surgeon to perform two separate excisions using different tools. It also requires the surgeon to manually support and guide the tools performing the excisions. Furthermore, some tools, such as an osteotome, are sometimes advanced under force, e.g., via a reciprocating sliding weight, or by striking with a mallet. Accordingly, when using an osteotome, the surgeon must oftentimes use one hand to securely hold and guide the osteotome, while using the other hand to advance the osteotome as described. In these cases, the surgeon must take particular care to follow the planned profile and to avoid tilting, pitching and/or deflection of the osteotome as it is being struck.

A potential advantage of the present device is in that it can be securely positioned in a self-supported manner at a surgical site, so as to guide a surgical instrument along a predetermined path, without deviation under force. Thus, the present device may relieve the surgeon of the need to simultaneously manually guide as well as advance the instrument. Rather, the surgeon need only hold down the device and advance the instrument.

Another potential advantage of the present device is in that, during nasal hump reduction, a complete hump, comprising both the cartilaginous and bony sections, may be removed in a single continuous pass along the predetermined path, while avoiding deflection and “jump” when the instrument encounters the junction between cartilage and bone. Thus, by using the present device, a surgeon may reduce the number of tools used and steps performed during rhinoplasty.

In some embodiments, the present device comprises a body and a handle. The device body comprises a guide channel configured for slidably receiving and engaging a surgical instrument, e.g., an osteotome or chisel, and guiding it along a desired application path. The device body may further comprise one or more distal support arms, and one or more adjustable support legs, configured for positioning the device at a desired position and/or orientation relative to the surgical site, and stabilizing the device against displacing forces exerted when the surgical instrument is being propelled, e.g., by striking it with a mallet. The adjustable support legs may be used for adjusting the path along which the surgical instrument is being guided.

In some embodiments, the device is made of a suitable material, e.g., a stainless-steel alloy. In other embodiments, the device may be configured as a disposable kit made of a suitable material, e.g., a polymer. In such embodiments, portion of the device, such as those portions which come in contact with a sliding osteotome, may be coated in a protective layer configured to resist abrasion.

In some embodiments, there is provided an osteotome guide system comprising an osteotome guide device as described above, at least one sensor, and a control unit. In some embodiments the osteotome guide system determines and/or monitors the position, location and/or orientation of the guide channel of the osteotome guide device in relation to a surgical site. In some embodiments, the system is configured to assist a surgeon to determine a position and/or orientation of the guide channel in relation to a surgical site to achieve a desired surgical result.

In some embodiments, the control unit comprises one or more of a processing module, a display, and a user interface module. In some embodiments, the system is configured to receive data inputted by a user. In some embodiments, the system is configured to receive data, such as an image, of a surgical site of a subject and determine a position, location, and/or orientation of the guide channel in relation to the surgical site. In some embodiments, the system allows a surgeon and/or user to determine the position, location, and/or orientation of the guide channel in a virtual reality and/or augmented reality display environment presenting the surgical site and/or the device. In some embodiments, the system models a virtual surgical site based on at least one of the data of a surgical site of a subject and/or other data inputted by a user.

In some embodiments, the system determines the position, location, and/or orientation of the guide channel in relation to the surgical site. In some embodiments, the system determines the position, location, and/or orientation of the guide channel according to a desired result of a procedure using the device on the surgical site. In some embodiments, the desired result is chosen by a surgeon and/or user from optional results depicted by the system. In some embodiments, the desired result is chosen by the surgeon and/or user manually within a virtual and/or augmented display environment presented by the system via, for example, a head-mounted display. In some embodiments, the desired result is chosen by the surgeon and/or user automatically within a virtual and/or augmented display environment presented by the system.

In some embodiments, the osteotome guide system is configured to measure its position, orientation, and/or location of the guide channel in relation to the surgical site of a subject in real time, such as during a procedure in which the device is used. In some embodiments, the system is configured to compare the measured position, orientation, and/or location, of the guide channel to the predetermined modeled guide channel location, orientation and/or position, and to notify a surgeon and/or user when the guide channel deviates from the predetermined position, configuration, location and/or orientation, e.g., by more than a specified threshold. In some embodiments, the system guides the surgeon and/or user to adjust at least one the location, orientation, and position of the guide channel, for example, by displaying the angle and/or distance of deviation of a the guide channel from the modeled position, location and/or orientation of the guide channel.

In some embodiments, the adjustment of the position, location and/or orientation, of the guide channel is manual, automatic or semi-automatic. In some embodiments, the adjustment of the device and/or parts thereof is based on the predetermined modeled position, location and/or orientation of the guide channel. In some embodiments, adjustment of the position, location and/or orientation of the guide channel is manual, automatic or semi-automatic, and replicates the predetermined modeled position, location and/or orientation of the guide channel in relation to the surgical site.

In some embodiments, the system is operable to provide, based on image data and/or other data descriptive of the subject's surgical site, instructions for automatically or semi-automatically manufacturing the surgical device and/or parts thereof using a variety of manufacturing techniques including, for example, 3D-printing, additive manufacturing, and/or machining (e.g., drilling, milling or otherwise three-dimensionally contouring one or more workpieces).

In some embodiments, the system is operable to provide a surgeon, based on at least image data and/or data which is descriptive of the subject's surgical site, personalized (for example, subject-oriented and/or surgeon-oriented) surgical guidance. In some embodiments, the system is operable to allow the manufacturing of a personalized surgical device based on image data and/or other data descriptive of the subject's surgical site and/or based on surgeon preferences.

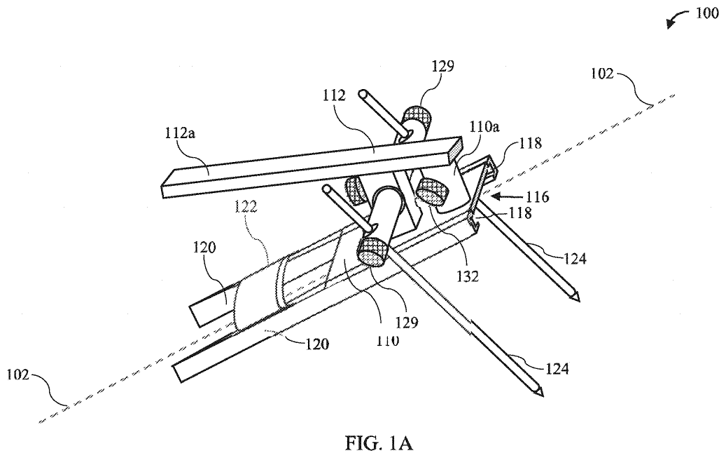

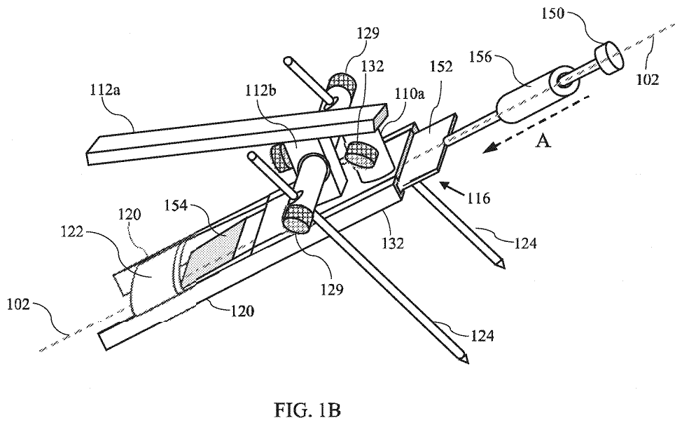

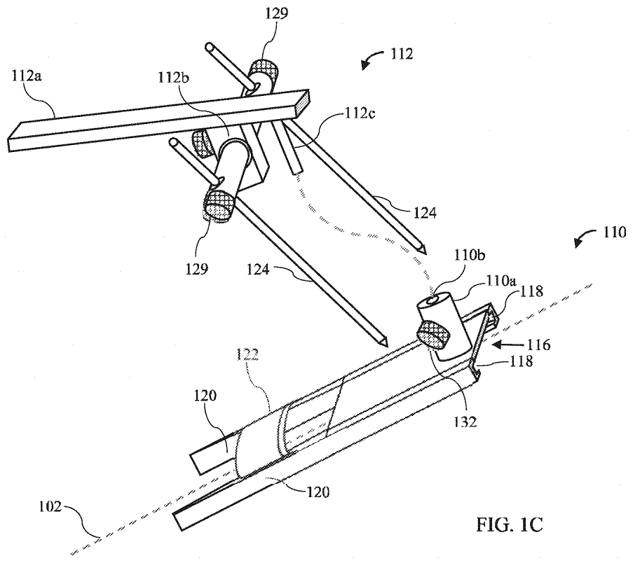

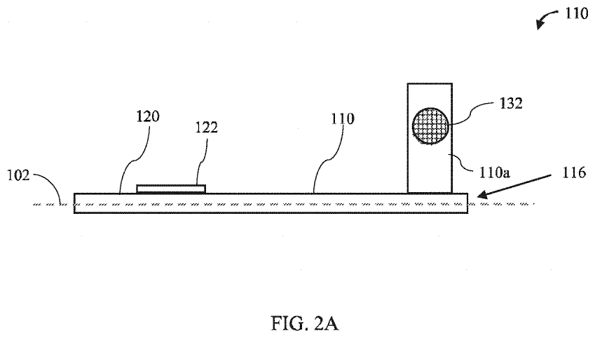

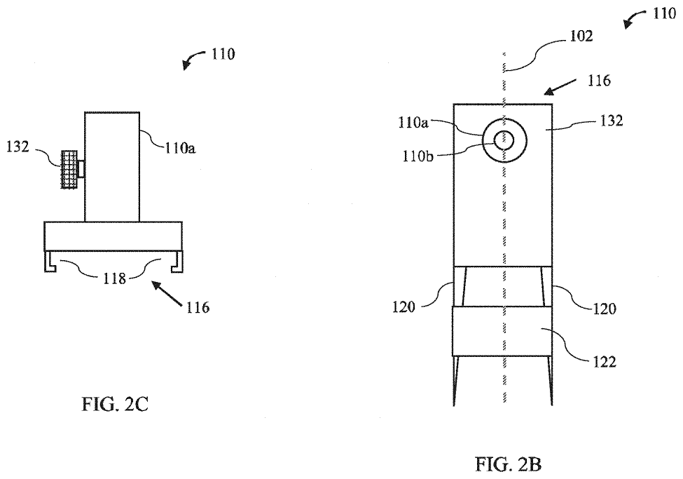

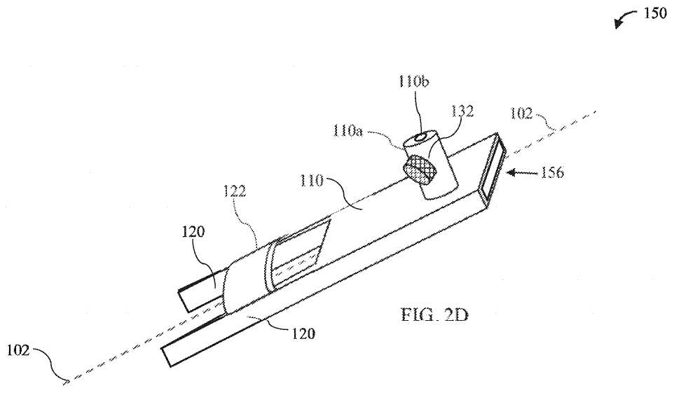

FIG. 1A illustrates an exemplary osteotome guide device 100 according to an embodiment of the present disclosure. In some embodiments, osteotome guide device 100 comprises a guide body 110 and a handle assembly 112. In certain embodiments, handle assembly 112 is a detachable handle assembly. FIG. 1C shows guide body 110 with handle assembly 112 in a detached state. FIGS. 2A-2C illustrate, respectively, side, top, and back views of guide body 110, with handle assembly 112 removed.

With reference to FIGS. 1A-2C, guide body 110 comprises guide channel 116 oriented along longitudinal axis 102. Guide channel 116 is dimensioned for slidably engaging and guiding an osteotome along at least a portion of its blade, so as to permit reciprocating movement of the osteotome along longitudinal axis 102, while resisting vertical movement, lateral movement, and rotation and/or pitching about a longitudinal axis or a transverse axis of the osteotome. In some embodiments, guide channel 116 is defined by a pair of opposing grooves 118 extending along longitudinal axis 102 on opposite sides of guide channel 116.

As shown in FIG. 1B, an osteotome 150 may be inserted through a proximal opening of guide channel 116, such that side edges of osteotome blade 152 are slidably engaged by grooves 118. The osteotome is then propelled slidably forward in the direction indicated by arrow A, until a cutting edge 154 is exposed at a distal end of guide channel 116. In some variations, grooves 118 may be adjustable grooves configured for receiving osteotomes of varying cross-sectional dimensions. In other embodiments, such as the one illustrated in FIG. 2D, guide body 150 comprises a guide channel 156 which is at least in part a hollow rectangular sheath extending within guide body 150 along longitudinal axis 102. In yet other embodiments, guide body 110 may comprise a changeable guide channel (not shown) configured for attaching to guide body 110. In such embodiments, a plurality of guide channels of varying cross-sectional dimensions may be configured for attaching to guide body 110, so as to engage diverse types and sizes of osteotomes. In some variations, the guide channels 116, 156 or portions thereof may be made of a material having a low coefficient of friction, or otherwise may be coated with a low friction compound, to facilitate the sliding of an osteotome therein.

With continued reference to FIGS. 1A-2C, in some embodiments, guide body 110 further comprises a stem portion 110a extending from guide body 110. Stem portion 110a is configured for removably attaching handle assembly 112 to guide body 110, e.g., by receiving an insert 112c (shown in FIG. 1B) of handle assembly 112 within bore 110b of stem 110a. In some embodiments, insert 112c is dimensioned to be tight-fitted within bore 100b. In other embodiments, a locking mechanism is provided comprising a threaded lock which extends through a threaded screw hole in stem 110a into bore 110b, such that a tip of lock screw 132 engages insert 112c. Thus, by tightening lock screw 132, insert 112c can be secured within bore 110b, and by loosening lock screw 132, insert 112c can be removed from bore 110b, thereby detaching handle assembly 112 from guide body 110.

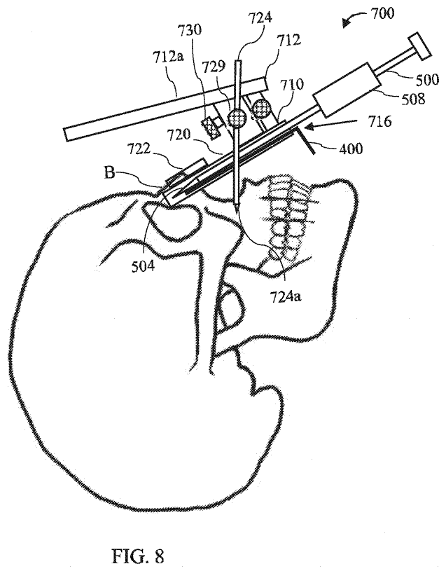

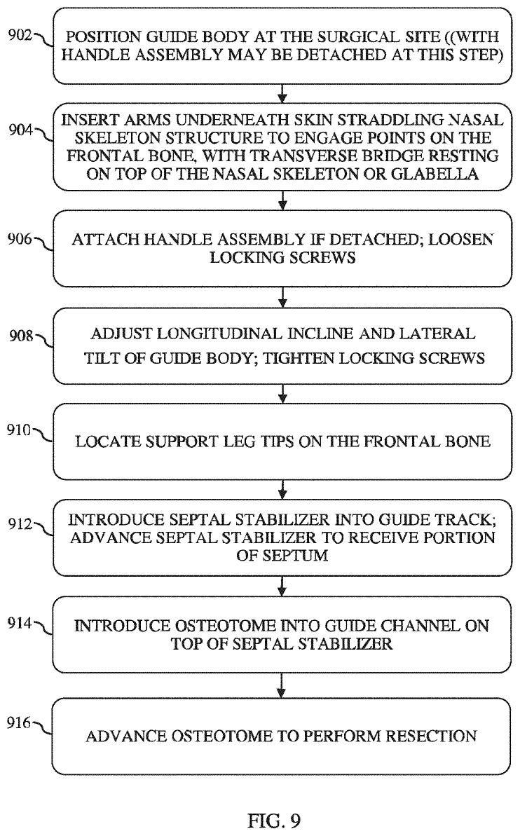

A pair of support arms 120 extend from opposite sides of guide channel 116 along longitudinal axis 102 from the distal end of guide body 110, such that the portion of the osteotome which protrudes from the distal end of the guide channel 116 is disposed between the support arms 120. With reference to FIG. 8, during an open or closed rhinoplasty procedure, distal ends of support arms 120 may be inserted underneath the skin (which, in open rhinoplasty, may be peeled back), and positioned so as to straddle the exposed nasal skeleton structure and engage relatively rigid points on the frontal bone. By being oriented along axis 102 and on the same path along which the osteotome is being propelled under impact, support arms 120 may help to counteract any forces and/or torque tending to shift or destabilize osteotome guide device 100. In some embodiments, the transverse distance between support arms 120 is adjustable, so as to accommodate a variety of surgical sites and/or anatomies during diverse procedures.

In some embodiments, a transverse bridge 122 extends between support arms 120. During open rhinoplasty, for example, bridge 122 may be positioned so as to rest on the top of the nasal skeleton structure. Bridge 122 may help to positively position the distal end of osteotome guide device 100, e.g., against the glabella, and ensure against a downward dislocation or slippage of the guide body 110, which may result in improper or excessive resection of the bone. In addition, bridge 122 helps in holding back the peeled skin flap from interfering in the procedure.

As shown in the exemplary embodiment depicted in FIG. 1A, device 100 is positioned on a surface of a bone 125 and contacts the surface of bone 125 in at least three points of contact marked by arrows designated reference numerals 170. A potential advantage of a minimum of three points of contact 170 is in the stability provided to device 100 guide channel 116/156 reducing the need for an operator to stabilize device 100 during operation.

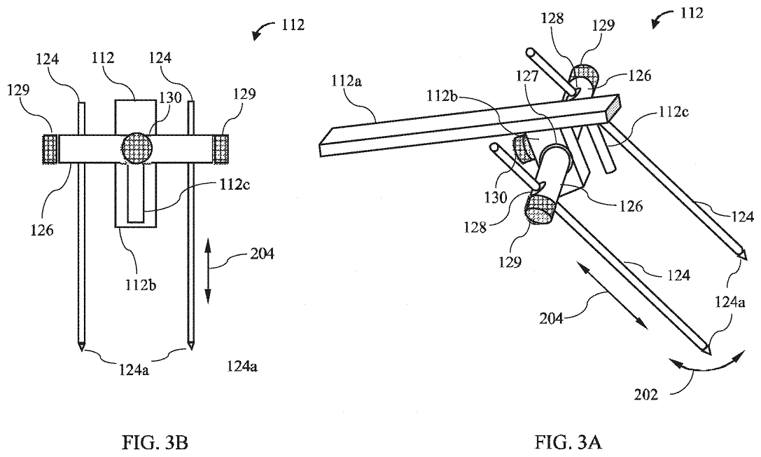

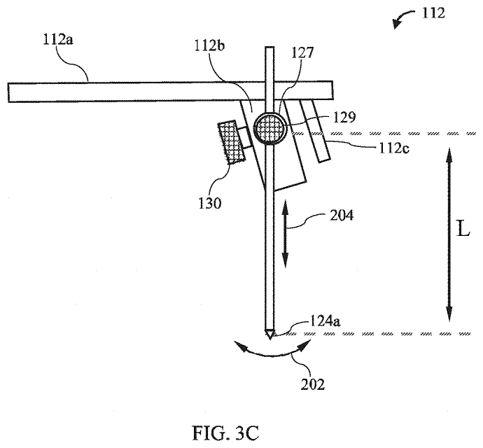

FIGS. 3A-3C illustrate, respectively, perspective, back, and side views of an exemplary embodiment of handle assembly 112. Handle assembly 112 comprises a grip portion 112a, a shank portion 112b, and insert 112c. In some embodiments, grip portion 112a is an elongated grip portion oriented along longitudinal axis 102. In some embodiments, handle assembly 112 may be removably attached to guide body 110, as explained above, by receiving insert 112c within bore 110b of guide body 110, and tightening lock screw 132 so as to selectively engage insert 112c within bore 110b. In certain embodiment, handle assembly 112 comprises a bore element to receive an insert element of guide body 110. In other embodiments, elements 112b, 112c of handle assembly 112 can be combined into a single element of handle assembly 112. In yet other embodiments, handle assembly 112 may be integrally formed with guide body 110, e.g., integrally molded with guide body 110 from a suitable material, such as a polymer.

A shaft 126 is rotatably received through a circular transverse opening 127 in shank portion 112b, such that shaft 126 is transversely disposed in relation to longitudinal axis 102. Each end of shaft 126 extends transversely beyond a corresponding transverse edge of guide body 110. In some embodiments, a threaded lock screw 130 extends into opening 127 through, e.g., a threaded hole in a side wall of shank portion 112b, such that a tip of lock screw 130 engages shaft 126. Lock screw 130 may thus provide for selectively locking shaft 126 within opening 127, such that shaft 126 may freely rotate about its axis when lock screw 130 is loosened, and is prevented from rotational movement when lock screw 130 is tightened.

A pair of support legs 124 are slidably received through corresponding openings 128 at each end of shaft 126. Support legs 124 may comprise tips 124a providing, e.g., a taper point which may engage the maxilla bone at the surgical site. A pair of threaded lock screws 129 are provided at opposing ends of shaft 126. Each lock screw 129 may extend through a threaded hole, e.g., on a respective the end face of shaft 126 and into the respective opening 128, for selectively engaging each of support legs 124 individually within its respective opening 128. Accordingly, each support leg 124 may freely slidably move as indicated by double-headed arrow 204 when its respective lock screw 129 is loosened, and is prevented from moving when its respective lock screw 129 is tightened.

With continued reference to FIGS. 3A-3C, in some embodiments, by loosening lock screw 130, support legs 124 may be rotatably adjusted about shaft 126 along arc 202. In some embodiments, shaft 126 comprises, e.g., two coaxial half-shafts individually rotatable, thus enabling individual rotational adjustment of each support leg 124 about a transverse axis of shaft 126.

In some embodiments, by loosening one or both of lock screws 129 and slidingly moving one or both of support legs 124 through respective openings 128 along double-headed arrow 204, a user may lengthen or shorten the effective length L shown in FIG. 3C (as measured from lock screw 129 to tip 124a) of each support leg 124 independently.

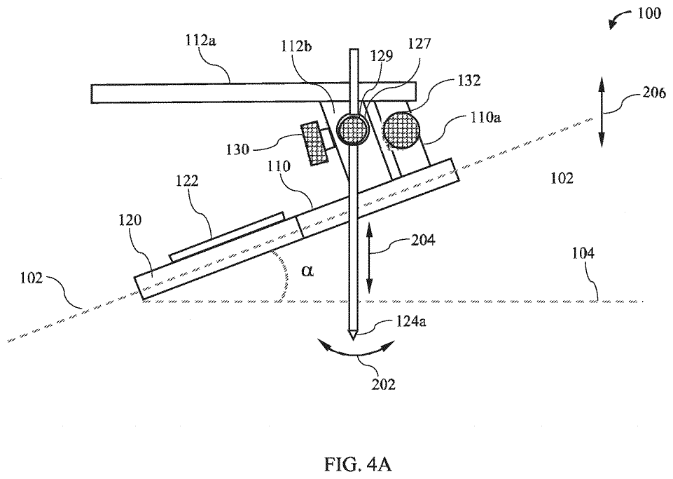

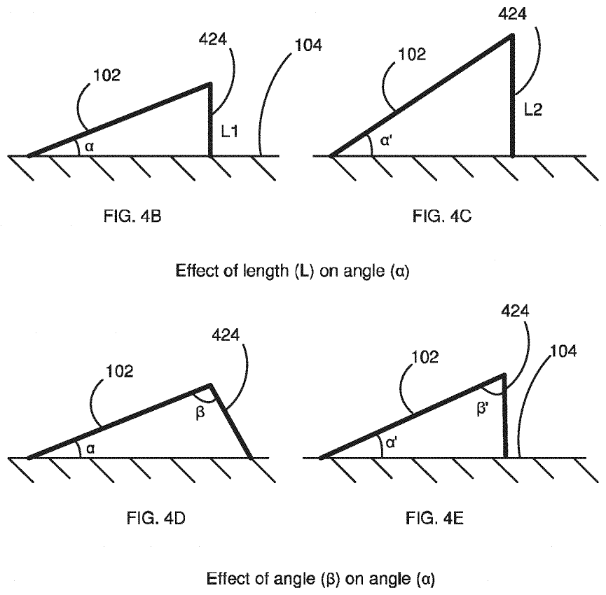

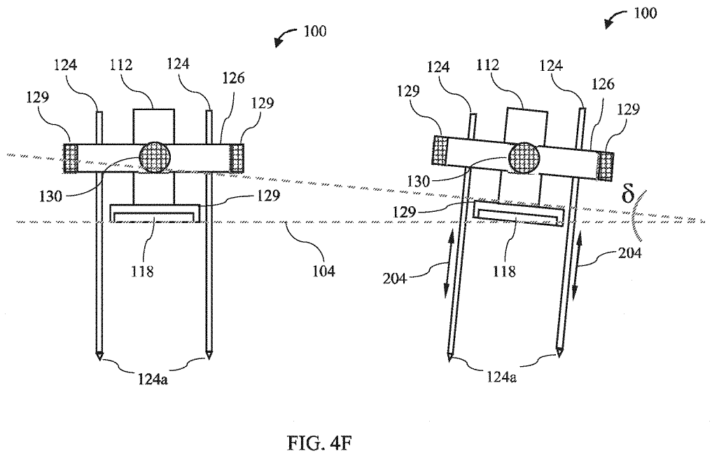

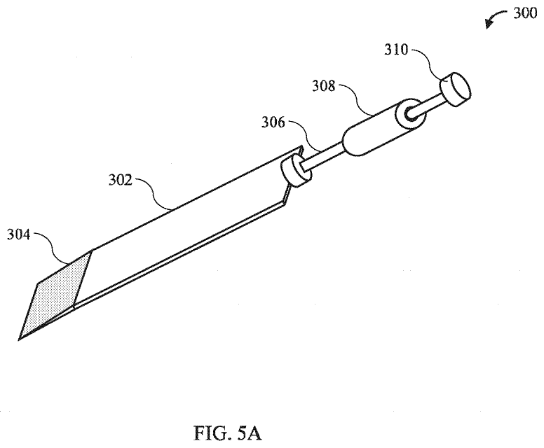

With reference to FIGS. 4A-4F, the user may use these adjustments of support legs 124 to accurately position and orient osteotome guide 100 at a surgical site. For example, in the case of rhinoplasty, the user may first position guide body 110 (with handle assembly 112 detached) at the surgical site, with arms 120 straddling the exposed nasal skeleton structure and engaging points on the frontal bone. The user may then attach handle assembly 112 by inserting insert 112c into bore 110b and locking lock screw 132. The user may then loosen lock screw 130 to allow adjustment of support legs 124 about shaft 126 along arc 202 (in tandem or individually for each support leg 124, depending on the embodiment). The user may also loosen one or both of lock screws 129 to lengthen or shorten the effective length L (shown in FIG. 3C) of each support legs 124 separately. Thus, the user is able to locate tips 124a of support legs 124 at desired respective points on the maxilla bone. The user is further able to raise or lower a proximal end of osteotome guide 100 in relation to a plane 104 defined by the surgical site, as indicated by double-headed arrow 204, thereby controlling an incline of guide channel 116 along longitudinal axis 102, which determines the angle of the insertion path of the osteotome and the depth of the cut, as indicated by angle α between longitudinal axis 102 and plane 104.

FIGS. 4B-4E are simplified illustrations of various optional adjustment possibilities provided by support legs 124. The angle between the insertion path of guide 100 and a plane 104 defined, for example, by a bone surface of the surgical site, is designated as an angle α. Angle α can be adjusted by lengthening and shortening the effective length of support legs 124, as shown in FIGS. 4B and 4C. For example, angle α can be increased by lengthening the effective length of support legs 124 from L1 to L2. Similarly, and as shown in the exemplary embodiments depicted in FIGS. 4D and 4E, angle α can also be changed by adjusting an angle β between support legs 124 and guide 100. As shown in the examples depicted in FIGS. 4D and 4E, angle α can be increased by reducing angle β to β′.

In some embodiments, both length L and angle β can be adjusted to compensate for the morphology of the surface of the bone as well as to adjust angle α.

With reference to FIG. 4F, in addition, by adjusting the individual effective length of each support leg 124, the user may control a transverse tilt of guide channel 116, as indicated in FIG. 4F by an angle δ between the transverse plane of guide channel 116 and plane 104 defined by the surgical site e.g., a surface of a bone.

Once a desired positioning and orientation of osteotome guide device 100 has been reached, the user may tighten lock screw 130 and lock screws 129, to retain the setting. Afterwards, the user may introduce an osteotome into a proximal opening of guide channel 116. The user may hold grip portion 112a of handle assembly 112 for added stability of osteotome guide device 100, and slidably propel the osteotome along the predetermined insertion path defined by guide channel 116 to perform the planned resection, without further need of manual guidance of the osteotome. In some embodiments, the resection is thus done by propelling the osteotome, e.g., using a sliding weight of the osteotome, in a single continuous pass along the predetermined insertion path.

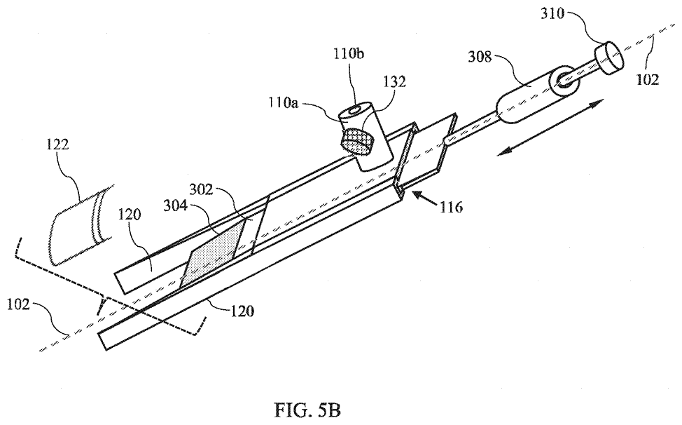

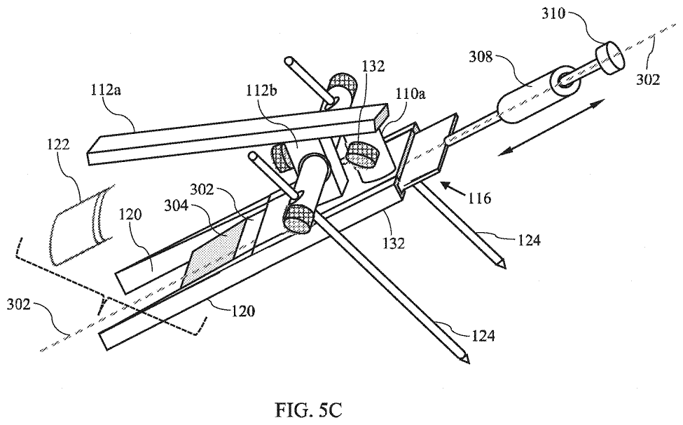

FIG. 5A illustrates an exemplary osteotome 300 of the type which may be used in conjunction with an osteotome guide of the present disclosure. Osteotome 300 comprises a blade 302 with a cutting edge 304, and a shank portion 306. In some embodiments, osteotome 300 comprises a reciprocating sliding weight or mallet 308 used to propel the osteotome. In other embodiments, a mallet may be used to strike cap 310.

FIG. 5B illustrates a perspective view of guide body 110 (with handle assembly 112 detached) with osteotome 300 received in guide channel 116. As can be seen, a lengthwise portion of blade 302 is engaged within guide channel 116 and can be moved reciprocatively along axis 102. Cutting edge 304 is exposed between support arms 120, underneath bridge 122 (shown in FIG. 3B separated from support arms 120). FIG. 5C illustrates a perspective view of guide body 110 with handle assembly 112 attached, with osteotome 300 received in guide channel 116.

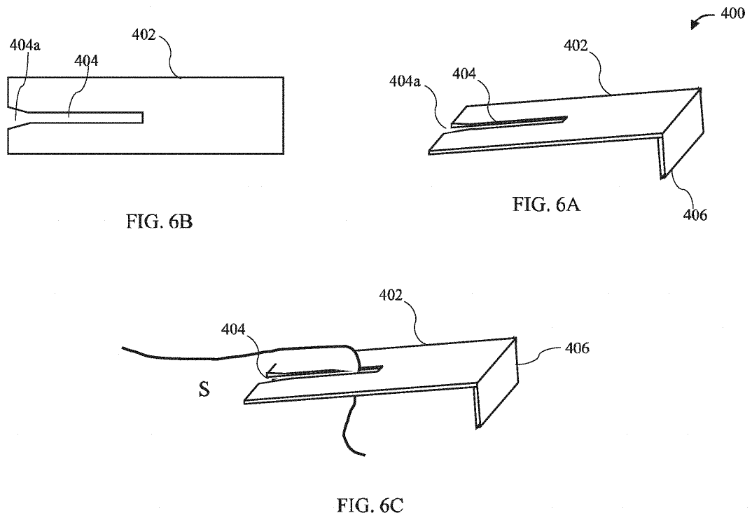

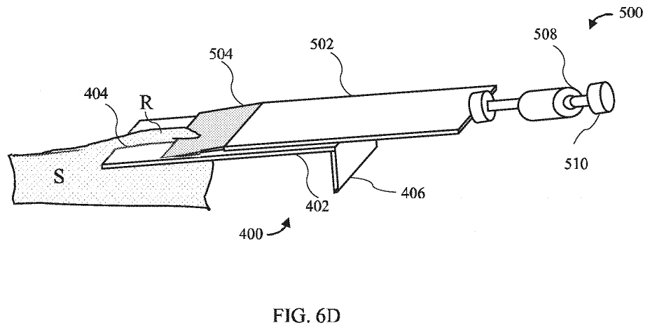

Reference is made to FIGS. 6A-6B showing, respectively, a perspective and top views of an exemplary septal stabilizer 400 for use in conjunction with the osteotome guide of the present disclosure. In some embodiments, septal stabilizer 400 comprises an elongated planar strip 402 having a centrally-located slot 404 extending from a distal transverse face thereof. In some embodiments, a forward end 404a of slot 404 broadens distally. Planar strip 402 may terminate at a proximal end with a stop portion configured to prevent septal stabilizer 400 from sliding into a proximal opening of the guide channel. In an exemplary embodiment shown in FIGS. 6A and 6C, the stop portion is a downward turned grip portion 406 extending from planar strip 402. In some embodiments, planar strip 402 is integrally formed from any suitable material, such as a stainless alloy or a polymer.

In some embodiments, as schematically shown in FIG. 6C, during open or closed rhinoplasty, septal stabilizer 400 is configured to be advanced, e.g., using grip portion 406, until a portion of exposed septum S is received within slot 404. In some embodiments, widened V-shaped mouth 404a is configured for facilitating the insertion of the exposed septum into slot 404. Septal stabilizer 400 thus stabilizes septum S against unwanted lateral movement and exposes only that portion of the septum which is planned for resecting. As schematically shown in FIG. 6D, in some embodiments, an osteotome 500, comprising shank portion 502, cutting edge 504, reciprocating weight 508, and cap 510, may be received above septal stabilizer 400 within a guide channel (not shown), and be slidably propelled along a surface of stabilizer 400 along an insertion path defined by the guide channel, to resect portion R of septum S exposed through slot 404. As noted above, in some embodiments, the resection by the osteotome is done in a single continuous pass along the insertion path.

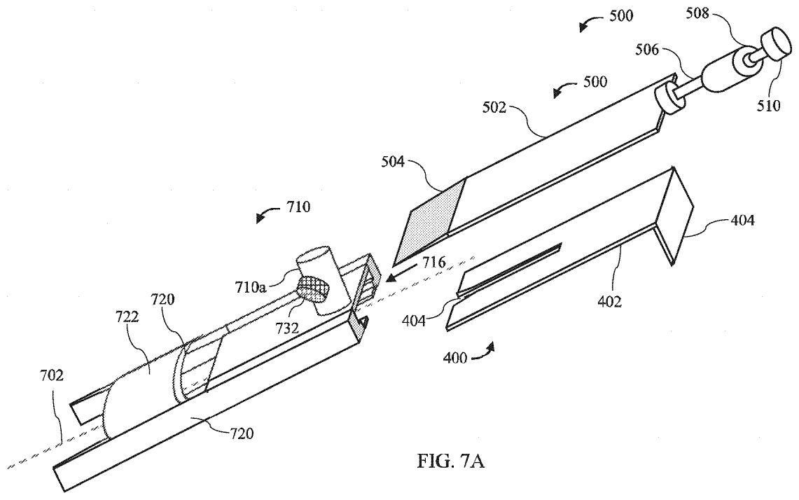

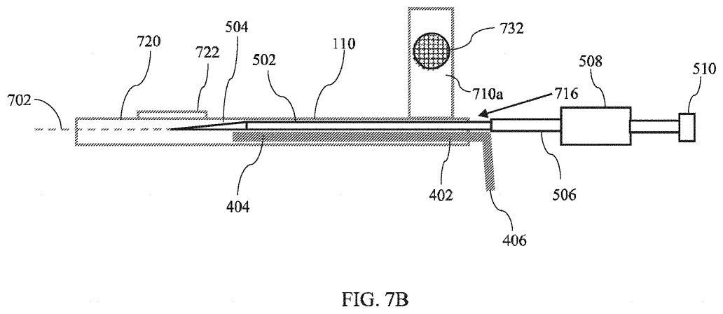

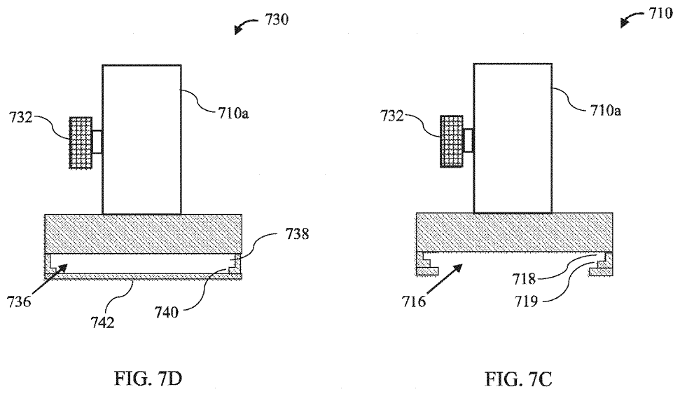

FIGS. 7A-7B illustrate the manner in which an osteotome 500 and septal stabilizer 400 may be received within a guide channel 716 of an osteotome guide 700, according to an embodiment of the present disclosure. Osteotome guide 700 may comprise stem portion 710a, support arms 720, transverse bridge 722, and lock screw 732. In some embodiments, guide channel 716 is oriented along longitudinal axis 702. Guide channel 716 is dimensioned to slidably engage and guide osteotome 500 along a top portion of guide channel 716, and to slidably engage and guide septal stabilizer 400 along a bottom portion of guide channel 716, such that osteotome 500 lies on top of, and is in slidable contact with, a top surface of septal stabilizer 400. As shown in FIG. 7B, in some embodiments, guide channel 716 may be defined by opposing stepped side grooves 718, 719. In some embodiments, opposing stepped side grooves 718 may be dimensioned to slidably engage the side edges of an osteotome, and opposing stepped side grooves 719 may be dimensioned to slidably engage the side edges of a septal stabilizer. In another embodiment illustrated in FIG. 7C, a guide channel 736 of guide body 730 is at least in part a hollow rectangular sheath extending within guide body 730. Guide channel 736 may also comprise opposing stepped side grooves 738, 740, as well as a bottom section 742.

FIG. 8 illustrates a rhinoplasty procedure using an osteotome guide according to an embodiment of the present disclosure. A method for performing a resection of the nasal bridge during rhinoplasty using osteotome guide 700 according to the present disclosure will be described below, with reference to FIG. 8 and to the block diagram in FIG. 9.

In a step 902, the user positions guide body 710 (optionally with handle assembly 712 detached) at the surgical site. In a step 904, the user advances arms 720 underneath the skin (which may be peeled back) astride the exposed nasal skeleton structure B, until arms 720 engage respective points on the frontal bone on either side of the nasal skeleton. Step 904 further comprises positioning bridge 722 on top of the nasal skeleton or the glabella, wherein bridge 722 helps retaining back the skin flap. In a step 906, the user attaches handle assembly 712 of guide body 710 (if detached earlier) and loosens (i) lock screw 730 to allow rotational adjustment of support legs 724 about a shaft (not shown) in tandem or individually for each support leg 724, depending on the embodiment, and (ii) one or both of lock screws 729, to lengthen or shorten the effective length of each support legs 724. In a step 908 the user may raise, lower or tilt a proximal end of osteotome guide 700, to control the longitudinal incline and lateral tilt of osteotome guide 700 in relation to the surgical site. Once a desired positioning and orientation has been reached in step 910, the user may tighten lock screws 729, 730. In a step 910, the user locates tips 724a of support legs 724 at desired respective points on the maxilla bone.

In a step 912, the user introduces septal stabilizer 400 into guide channel 716 and advances it so as to receive a portion of exposed nasal skeleton B in a forward slot of septal stabilizer 400 (not shown). In a step 914, the user introduces osteotome 500 into guide channel 716. In a step 916, the user grips handle 712a with one hand and propels osteotome 500 along the insertion path defined by guide channel 716 with the other hand, e.g., by using sliding weight 508, or by striking osteotome 500 with a mallet. Osteotome 500 is thus propelled along the predetermined insertion path to perform the resection in a single continuous pass, without further need of manual intervention in adjusting its trajectory.

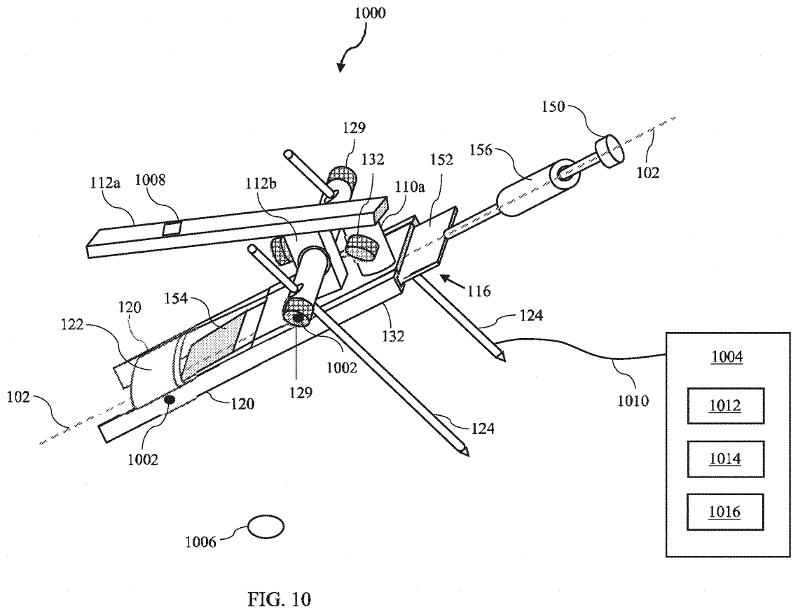

Reference is made to FIG. 10, which is an osteotome guide system 1000 according to some embodiments of the present disclosure. In some embodiments, the osteotome guide system 1000 comprises an osteotome guide device and a control unit 1004. In some embodiments, the osteotome guide device comprises at least one sensor 1002 in communication with a control unit 1004. In some embodiments, control unit 1004 comprises one or more of a processing module 1012, a display 1014, and a user interface module 1016.

In some embodiments, the system 1000 is configured to assist a surgeon in determining a positioning, location and/or orientation of the guide channel 116 in relation to a surgical site to achieve a desired surgical result. In some embodiments, the system 1000 determines the position, location, and/or orientation of the guide channel 116 in relation to a surgical site during a preparation stage in which the system 1000 models optional procedure results using the device.

In some embodiments, one or more sensors 1002 are configured to measure a position, location and/or orientation of the guide channel 116 in relation to a reference point. In some embodiments, the reference point is at or around a surgical site.

In some embodiments, the control unit 1004 comprises data comprising the location of the guide channel 116 in relation to one or more sensor 1002 and/or marker 1006.

In some embodiments, the at least one sensor 1002 is positioned on, attached to and/or embedded within portions of the device, such as, but not limited to, the support legs 124, lock screw 129, osteotome blade 152, guide channel 156, osteotome 150, support arms 120, bridge 122, handle assembly 112, and lock screw 132. In some embodiments, at least one sensor 1002 is positioned on or around the surgical site.

In some embodiments, the at least one sensor 1002 is one or more of an accelerometer, proximity sensor, capacitive sensor, optic sensor, IR sensor, sound sensor, hall effect sensor, ultrasonic sensor, touch sensor, vibration sensor, and/or the like. In some embodiments, the system 1000 comprises a plurality of sensors 1002 which are in communication with each other.

In some embodiments, a plurality of the sensors 1002 determine the distances between each other. In some embodiments, a plurality of the sensors 1002 determine the angles between each other and/or in relation to the horizon and/or relative to one or more of the subject's body axes and/or planes (e.g., the sagittal axis and/or the sagittal plane).

In same embodiments, the system 1000 comprises a marker 1006. In some embodiments, the marker 1006 is configured to be placed onto the bone of a subject, such as on the maxilla, skin of a subject, a tooth, and other reference points, such as the surgical bed and locations around the surgical site. In some embodiments, the sensor 1002 recognizes the marker 1006.

In some embodiments, the marker 1006 is a reference point used to determine the position, location, and/or orientation of the guide channel 116 relative to the subject's surgical site. In some embodiments, the marker 1006 is placed onto a predetermined position in reference to a surgical site, such that the, position, location, and/or orientation of the guide channel 116 is calculated with reference to the surgical site. In some embodiments, the control unit 1004 calculates the position, location, and/or orientation of the guide channel 116 with reference to the surgical site using the at least one sensor 1002 and the at least one marker 1006.

In some embodiments, the at least one sensor 1002 is in communication with a control unit 1004. In some embodiments, the at least one sensor 1002 is coupled to the system 1000 and/or control unit 1004 via one or more of electrical cable 1010, Bluetooth, Wi-Fi, and/or wired and/or wireless communication technology. In some embodiments, the control unit 1004 is integral to the osteotome guide device.

In some embodiments the control unit 1004 determines and/or monitors the position, location and/or orientation of the osteotome guide device in relation to a surgical site. In some embodiments the control unit 1004 determines and/or monitors the position, location and/or orientation of the guide channel 116 using signals of the at least one sensor 1002. In some embodiments, the control unit 1004 is configured to receive an image of a surgical site and determine the a position, location and/or orientation of the guide channel 116 in relation to the surgical site within a virtual environment. In some embodiments, the control unit 1004 comprises a processing module 1012 configured to receive and/or analyze at least one image of a subject, such as, but not limited to, a digital image, x-ray image, Magnetic resonance imaging (MRI) scan, computed tomography (CT) scan.

In some embodiments, the processing module 1012 calculates and/or determines possible positions, locations and/or orientations of the guide channel 116 in relation to the surgical site. In some embodiments, the processing module 1012 calculates and/or determines possible positions, locations and/or orientations of the guide channel 116 within a virtual environment. In some embodiments, the display 1014 displays possible results of usage for the device with specific positions, locations and/or orientations of the guide channel 116 in relation to the surgical site.

In some embodiments, a desired result is chosen by the surgeon and/or user manually within a virtual environment presented by the system. In some embodiments, a desired result is chosen by the surgeon and/or user automatically within a virtual and/or augmented environment presented by the system. In some embodiments, the virtual environment is displayed onto the display 1014, and in some embodiments, the virtual or augmented environment depicts the patient-specific surgical site and the device. In some embodiments, the virtual or augmented environment presents optional outcomes of procedures using the device on the patient specific surgical site. In some embodiments, the optional outcomes are presented onto the display 1014.

In some embodiments, the processing module 1012 models position, location and/or orientation of the guide channel 116 for an outcome of a procedure using the device chosen by a surgeon and/or user.

In some embodiments, the display 1014 presents a surgeon and/or user with modeled positions, locations and/or orientations of the guide channel 116 in a virtual environment. In some embodiments, In some embodiments, the virtual environment includes at least one of the surgical site and the device.

In some embodiments, at least one of the display 1014, the indicator 1008, the processing module 1012, and the user interface module 1016 are coupled via one or more of electrical cable 1010, Bluetooth, Wi-Fi, and/or wired and/or wireless communication technology. In some embodiments, the guide is integrated onto the system 1000, such as on the handle assembly 112.

In some embodiments, the display 1014 displays optional outcomes of a procedure within a virtual or augmented environment using the device in different positions, locations and/or orientations in relation to the surgical site. In some embodiments, the user interface module 1016 allows a surgeon to choose a desired outcome of a procedure using the device.

In some embodiments, the processing module 1012 determines the required position, location and/or orientation of the guide channel 116 of the device in relation to a surgical site of a subject for a specific desired outcome of a procedure.

In some embodiments, the virtual or augmented environment presented to the surgeon and/or user via the display 1014 comprises optional procedure results of the device on a surgical site. In some embodiments, the surgical site of the virtual or augmented environment is based, at least in part, on an image received by the control unit 1004.

In some embodiments, the surgeon and/or user chooses a modeled result from the optional procedure results presented by the display 1014. In some embodiments, the control unit 1004 provides at least one of a required position, location and/or orientation of the guide channel 116 and/or other portion of the device in relation to the surgical site for the chosen modeled result. In some embodiments, applying the modeled position, location and/or orientation of the guide channel 116 and/or other portion of the device in relation to the surgical site during procedure allows achieving the modeled result.

In some embodiments, the indicator 1008 is one or more of a visual, tactile and/or auditory display unit (e.g., light bulb, audio emitting device, haptic output and/or the like), configured to alert (e.g., light up, display a notice, emit an audio signal, vibration alert) a user. For example, in some embodiments, the indicator 1008 alerts a user during a procedure, when the device is deviated from the determined modeled position, location and/or orientation, e.g., by more than a specified threshold. In some embodiments, the indicator 1008 is integral to the control unit 1004. In some embodiments, such as depicted by FIG. 10, the indicator 1008 is positioned an the device. In some embodiments, the deviation threshold is predetermined by a user.

In some embodiments, the system 1000 alerts the user of a deviation higher than a predetermined threshold. In some embodiments, the alert is one or more of an auditory alert, a visual alert, a textual alert, and a haptic alert.

In same embodiments, the deviation is one or more of degrees of an angle deviation, the distance of at least a portion of the device in relation to the surgical site, and a deviation between the modeled position, location, and/or orientation of the guide channel 116 and/or other portion of the device in comparison to the determined position, location and/or orientation of the guide channel 116 and/or other portion of the device using one or more sensor 1002. In some embodiments, the system 1000 monitors the position, location, and/or orientation of the guide channel 116 and/or other portion of the device in relation to the surgical site in real time (e.g., during a surgical procedure in which the device is used).

In some embodiments, the control unit 1004 notifies the user the adjustments which are needed for alignment of the device. In some embodiments, the control unit 1004 notifies the user of adjustments (e.g. a change in angle, distance, spatial orientation, and/or location) needed to be made to the guide channel 116 and/or other portion of the device for a procedure to result with a desired outcome chosen by a user.

In some embodiments, the system guides the surgeon and/or user to adjust (e.g., re-configure and/or re-position) the device and/or parts thereof such that the deviation of the new position, location and/or orientation of the guide channel 116 and/or other portion of the device is lower than the predetermined threshold deviation value. In some embodiments, the system guides the surgeon by displaying, for example, positional parameter values such as, for example, the angle and/or distance of deviation of the guide channel 116 and/or a portion of the device from the modeled position, location and/or orientation of the guide channel 116 and/or portion of the device. In some embodiments, the system guides the surgeon and/or user to adjust (e.g., re-position and/or reconfigure) the device and/or guide channel 116 to the modeled position, location and/or orientation in real time and/or near-real time (for example, during a surgical procedure in which the device is used).

For example, in some embodiments, the control unit 1004 notifies a user of a required change of one or more of the angle α between the guide 100 and a plane 104 defined, for example, by a bone surface of the surgical site, an angle β between support legs 124 and guide 100, the angle δ between the transverse plane of guide channel 116 and plane 104 defined by the surgical site e.g., a surface of a bone, and the spatial orientation of at least one portion of the device.

In some embodiments, the at least one marker 1006 is positioned on, attached to and/or embedded within portions of the device, such as, but not limited to, the support legs 124, lock screw 129, osteotome blade 152, guide channel 156, osteotome 150, support arms 120, bridge 122, handle assembly 112, and lock screw 132. In some embodiments, the at least one marker 1006 is positioned at the surgical site, for example, at the base of the nose of a subject, a tooth of a subject, and the maxilla of a subject. In some embodiments, the at least one marker 1006 is detachable. In some embodiments, the at least one marker 1006 is disposable.

In some embodiments, the at least one sensor 1002 is positioned to detect said at least one marker 1006. In some embodiments, the at least one sensor 1002 is positioned at the surgical site. In some embodiments, the at least one sensor 1002 is positioned around the surgical site. For example, in some embodiments, the sensor 1002 is positioned at the base of the nose of a subject, a tooth of a subject, and the maxilla of a subject. In some embodiments, at least one sensor 1002 and/or at least one marker 1006 are positioned on at least one reference point.

In some embodiments, at least one sensor 1002 detects at least one marker 1006 and identifies the special orientation and/or location of the device in relation to the surgical site.

In some embodiments, there are a plurality of markers 1006 on the device. In some embodiments, at least one sensor 1002 detects the plurality of markers 1006 on the device. In some embodiments, the sensor 1002 identifies the position, location, and/or orientation of the guide channel 116 and/or other portion of the device in 3D space in respect to the surgical site based on the relation between the plurality of markers 1006 on the device and/or on the surgical site.

Any digital computer system, module and/or engine exemplified herein can be configured or otherwise programmed to implement a method disclosed herein, and to the extent that the system, module and/or engine is configured to implement such a method, it is within the scope and spirit of the disclosure. Once the system, module and/or engine are programmed to perform particular functions pursuant to computer readable and executable instructions from program software that implements a method disclosed herein, it in effect becomes a special purpose computer particular to embodiments of the method disclosed herein. The methods and/or processes disclosed herein may be implemented as a computer program product that may be tangibly embodied in an information carrier including, for example, in a non-transitory tangible computer-readable and/or non-transitory tangible machine-readable storage device. The computer program product may directly loadable into an internal memory of a digital computer, comprising software code portions for performing the methods and/or processes as disclosed herein.

Additionally or alternatively, the methods and/or processes disclosed herein may be implemented as a computer program that may be intangibly embodied by a computer readable signal medium. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a non-transitory computer or machine-readable storage device and that can communicate, propagate, or transport a program for use by or in connection with apparatuses, systems, platforms, methods, operations and/or processes discussed herein.

The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed an one computer or on multiple computers at one site or distributed across multiple sites and interconnected by one or more communication networks.

These computer readable and executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The term “processing module” as used herein in the context of computerized functionalities may comprise one or more computer modules. Exemplarily, a module may be a self-contained hardware and/or software component that interfaces with a larger system. A module may comprise a machine or machines executable instructions. A module may be embodied by a circuit and/or a controller programmed to cause the system to implement the method, process and/or operation as disclosed herein. For example, a module may be implemented as a hardware circuit comprising, e.g., custom Very Large Scale Integrated (VLSI) circuits or gate arrays, an Application-specific integrated circuit (ASIC), off-the-shelf semiconductors such as logic chips, transistors, and/or other discrete components. A module may also be implemented in programmable hardware devices such as field programmable gate arrays, programmable array logic, programmable logic devices and/or the like.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A device for guiding a surgical instrument at a surgical site, comprising:
at least one guide channel sized and fitted to slidingly accommodate a surgical instrument, said guide channel defining a guide longitudinal axis; and
at least one support leg coupled to said guide channel and defining a support leg longitudinal axis,
wherein an angle (α) of said guide longitudinal axis in respect to a surface of a surgical site is determined by at least one of (i) a length (L) of said at least one support leg, and (ii) an angle (β) between said guide longitudinal axis and said at least one support leg longitudinal axis.

2. The device of claim 1, wherein said at least one support leg is coupled to said guide channel through a coupling configured to allow angular adjustment of said at least one support leg in relation to said guide channel.

3. The device of claim 2, wherein said coupling has a locked state in which said at least one support leg is locked in a desired position relative to said guide channel, and an unlocked state in which said at least one support leg is able to be moved relative to said guide channel about said coupling.

4. (canceled)

5. The device of claim 2, wherein said coupling comprises a shaft disposed transversely in relation to said guide channel, wherein said shaft is rotatable about its longitudinal axis, and wherein said shaft is lockable in a fixed position to prevent rotation of said shaft relative to said guide channel.

6. (canceled)

7. The device of claim 2, wherein said coupling is configured to slidingly receive the at least one support leg and to slide along at least a portion of said length (L) of the at least one support leg, wherein said coupling is lockable is a position along said at least a portion of said length (L) of the at least one support leg.

8-13. (canceled)

14. The device of claim 1, further comprising at least one support arm rigidly coupled to said guide channel.

15. The device of claim 14, wherein said at least one support arm extends distally from a side of a distal end of said guide channel along said guide longitudinal axis.

16. The device of claim 14, further comprising a transverse projection extending laterally from said at least one support arm.

17. The device of claim 1, further comprising two opposed elongated support arms extending distally from opposite sides of a distal end of said guide channel along said guide longitudinal axis.

18. (canceled)

19. The device of claim 1, wherein said guide channel comprises a pair of opposing grooves extending along said guide longitudinal axis, wherein said grooves are configured to slidably engage corresponding side edges of said surgical instrument.

20-23. (canceled)

24. The device of claim 1, wherein said guide channel is sized and fitted to slidingly accommodate a first surgical instrument and a second surgical instrument, wherein each of said first surgical instrument and said second surgical instrument is disposed along said guide longitudinal axis.

25. The device of claim 24, wherein said guide channel comprises a pair of opposing two-stepped grooves extending along said guide longitudinal axis, wherein a first step of said pair of opposing two-stepped grooves is sized and fitted to slidingly accommodate said first surgical instrument, and wherein a second step of said pair of opposing two-stepped grooves is sized and fitted to slidingly accommodate said second surgical instrument.

26. (canceled)

27. The device of claim 24, wherein said second surgical instrument is a septal stabilizer.

28. The device of claim 27, wherein said septal stabilizer is configured for stabilizing a nasal septum during open rhinoplasty, said septal stabilizer comprising an elongated planar strip having a proximal end and a distal end, said distal end having a centrally-located slot configured to receive at least a portion of the nasal septum.

29. The device of claim 28, wherein said opening of said slot broadens distally.

30. The device of claim 27, wherein said septal stabilizer further comprises a stop portion configured to prevent at least a portion of the septal stabilizer from sliding into a proximal opening of said guide channel.

31. A device for guiding a surgical instrument at a surgical site, comprising:
a handle;
a guide channel sized and fitted to slidingly accommodate a surgical instrument, said guide channel defining a guide longitudinal axis;
two opposed elongated support arms extending distally from opposite sides of a distal end of said guide channel along said guide longitudinal axis;
a bridge disposed transversely between said support arms, wherein said bridge is attached to a top portion of each of said support arms; and
a pair of support legs disposed on either side of said body, wherein each of said support legs is pivotable about an axis that is transverse to the guide longitudinal axis, and wherein a length of each support leg is adjustable.

32-68. (canceled)

69. A system comprising:
a device for guiding a surgical instrument at a surgical site comprising:
at least one guide channel sized and fitted to slidingly accommodate a surgical instrument, said guide channel defining a guide longitudinal axis, and
at least one support leg coupled to said guide channel and defining a support leg longitudinal axis, wherein an angle (α) of said guide longitudinal axis in respect to a surface of a surgical site is determined by at least one of (i) a length (L) of said at least one support leg, and (ii) an angle (β) between said guide longitudinal axis and said at least one support leg longitudinal axis;
a control unit; and
at least one sensor in communication with said control unit;
wherein said at least one sensor is positioned to determine at least one of a location, a position, and an orientation of said guide channel in relation to at least one reference point.

70-71. (canceled)

72. The system according to claim 69, wherein said at least one reference point comprises a marker associated with said at least one sensor, and wherein said determining comprises measuring one of a distance, direction, alignment, orientation, and angle between said at least one sensor and said marker.

73. (canceled)

75. The system according to claim 69, wherein said control unit is configured to model a surgical procedure with respect to said surgical site using said device based, at least in part, on an image of the surgical site received by said control unit, and wherein said modelling determines a modeled location, position, and orientation of said guide channel and/or said device in relation to said surgical site.

76-81. (canceled)
